(19) United States
(12) Patent Application Publication
(10) Pub. No.: US 2021/0239670 A1
(43) Pub. Date: Aug. 5, 2021

(54) METHOD OF CONFIGURING AN AIR MONITORING SYSTEM

(71) Applicant: SimpleWater, Inc., Berkeley, CA (US)

(72) Inventors: John H. Pujol, Berkeley, CA (US); Matthew P. Vannucci, Oakland, CA (US)

(21) Appl. No.: 16/782,663

(22) Filed: Feb. 5, 2020

(51) Int. Cl. G01N 33/00; G05B 13/02

(52) U.S. Cl. CPC G01N 33/0075; G05B 13/024; G01N 33/007

(57) ABSTRACT

An occupant installed air monitoring system for monitoring indoor air quality comprises one or more air-quality sensors. The sensors are installed by an occupant following instructions provided by the air monitoring system. The effectiveness of the installation is improved when the sensor arrangement is changed by the occupant following further instructions that the air monitoring system generates based on sensor readings. The effectiveness is further improved when the air monitoring system receives information about occupant activities that affect air quality. Gamification techniques are used to achieve enhanced occupant participation and compliance.

### TECHNICAL FIELD

The present disclosure relates to systems for monitoring indoor air quality, and, more particularly, to methods for configuring a system for monitoring indoor air quality.

### REFERENCE CITED

“Ambient air pollution: A global assessment of exposure and burden of disease,” World Health Organization (2016), ISBN: 9789241511353.

### BACKGROUND

Indoor air quality affects the health, well-being, and productivity of occupants of an indoor environment. Globally, health issues from air quality problems are one of the leading causes of mortality. According a 2016 report by World Health Organization, outdoor and indoor air pollution account for a significant portion of all global deaths. Indoor air pollution negatively affects occupant productivity, well-being, and comfort.

Knowledge of the quality of indoor air is important for occupants of an indoor environment. Also, knowledge of indoor air quality can be used to advantage for adjusting the operating parameters of systems that provides heating, ventilation, and air conditioning (HVAC) in indoor environments.

A system for monitoring indoor air quality (hereinafter, air monitoring system) typically comprises one or more sensors that sense one or more parameters relevant to air quality (hereinafter, air quality sensors). Parameters relevant to air quality comprise many types of parameters that are relevant to assessing air quality. For example, parameters relevant to air quality may comprise: air temperature, humidity, radiant temperature, operative temperature, pressure, ionizing radiation, air movement, suspended particulate matter, aerosols, chemicals, reactive species, ozone, NOₓ, SOₓ, CO, CO₂, radicals, metals, and any other parameters that may be relevant to assessing air quality.

An air quality sensor can be configured to sense one or more air quality parameters to yield one or more sensor readings that reflect the sensed parameters. An air monitoring system can collect sensor readings from one or more sensors. The air monitoring system can process such readings to yield an assessment of air quality and, possibly, to initiate appropriate actions in response to the assessment. For example, a simple thermostat can be regarded as an air monitoring system that monitors air temperature to yield an assessment of occupant thermal comfort (the air quality). If thermal comfort is deemed inadequate (i.e., the temperature is lower than the set point) the thermostat initiates corrective action (by activating a heating functionality in the HVAC system).

Modern technology makes it possible to have a large number of low-cost sensors that can sense a large number of parameters; however, the assessment of air quality that can be derived from sensor readings is also affected by other important factors. For example, the position of a sensor in an indoor environment strongly affects the relevance of the sensor's readings. Also, when multiple sensors are deployed in an indoor environment, the arrangement of the sensors is important. In particular, the arrangement of the sensors relative to one another affects the ability of an air monitoring system to relate readings from different sensors to one another. Furthermore, the arrangement of the sensors relative to different zones of the indoor environment affects the ability of the air monitoring system to relate sensor readings to air quality in those zones.

In the prior art, effective positioning and arranging of sensors can be achieved by employing an expert installer. The expert installer is an individual with specialized knowledge and expertise who deploys sensors in an arrangement that is effective for air quality monitoring. Furthermore, the expert installer can configure the air monitoring system such that the system is aware of the arrangement of sensors relative to one another, relative to the zones of the indoor environment, and also relative to elements that can affect sensor effectiveness such as, for example, doors, windows, fireplaces, and other elements relevant to indoor air quality and/or sensor functionality.

One problem with employing an expert installer is the increased cost of installing and configuring the air monitoring system, which is due to the need to compensate the expert installer. Furthermore, the expert installer can only assess the conditions of the indoor environment at the time of sensor deployment. If indoor conditions change, it may be necessary to hire the expert installer again to reconfigure the air monitoring system based on the new conditions.

In the prior art, some indoor air monitoring systems do not require expert installation. For example, such systems might provide installation instructions to an occupant of an indoor environment. The instructions can direct the occupant to position and arrange sensors so as to achieve effective air quality monitoring. However, the effectiveness of the resulting arrangement depends on the ability of the occupant to correctly understand and implement the instructions, and on the clarity and thoroughness of the instructions themselves. Generally, such occupant-installed air monitoring systems do not achieve the same level of effectiveness as expert-installed systems.

It would be advantageous to have an occupant-installed air monitoring system that can achieve or even exceed the level of effectiveness achieved by expert-installed systems.

### SUMMARY

Embodiments of the present disclosure provide occupant-installed air monitoring systems that achieve an enhanced level of effectiveness compared to the prior-art. Their effectiveness can be close to or even exceed the effectiveness of expert-installed air monitoring systems. In some embodiments, an occupant initially installs one or more sensor, as directed by the air monitoring system. Then, the air monitoring system collects readings from the sensors and, based on those readings, may direct the occupant to rearrange the sensors as needed to improve system effectiveness. This may be repeated, as necessary.

In some embodiments of the present disclosure, the air monitoring system can improve its effectiveness by receiving information about certain activities performed by occupants of the indoor environment. For example, cooking affects air quality in various ways. Knowing that cooking activity is occurring enables the air monitoring system to correlate sensor readings with the activity and, thereby, improve its effectiveness.

The air monitoring system can receive information about occupant activity from the occupants themselves. Occupants can interact with the air monitoring system, for such a purpose, in different ways: (i) The air monitoring system can prompt occupants for information about ongoing activities; for example, if a particular pattern of sensor readings is detected, the air monitoring system can issue a prompt asking occupants for information. Alternatively, the air monitoring system can (ii) explicitly request that occupants perform a specific activity so as to see how sensor readings are affected by the activity. Also, the air monitoring system can (iii) provide a user interface for occupants to voluntarily communicate information about ongoing activities without a prompt or request from the air monitoring system.

Occupants might find it onerous to participate in (i), (ii), or (iii). Some embodiments of the present disclosure may encourage occupant participation via the use of gamification techniques. Gamification is the application of game-design elements and game principles in non-game contexts to improve user engagement, learning, and generally enhance user participation. Some embodiments of the present disclosure can use gamification techniques to enhance interactions with occupants so as to achieve more effective occupant participation in (i), (ii), and (iii), as well as in the installation, arrangement, and/or rearrangement of the sensors.

### BRIEF DESCRIPTION OF THE DRAWINGS

FIG. 1 depicts part of an installation of an occupant-installed air monitoring system.

FIG. 2 is a flow diagram of a process 200 in the prior art for configuring an occupant-installed air monitoring system.

FIG. 3 is a flow diagram of a process 300 for configuring an occupant-installed air monitoring system with improved effectiveness in accordance with a first illustrative embodiment of the present disclosure.

FIG. 4 is a flow diagram of a process 400 for improving the effectiveness of an air monitoring system in accordance with a second illustrative embodiment of the present disclosure.

FIG. 5 is a flow diagram of a process 500 for improving the effectiveness of an air monitoring system in accordance with a third illustrative embodiment of the present disclosure.

FIG. 6 is a flow diagram of a process 600 for improving the effectiveness of an air monitoring system in accordance with a fourth illustrative embodiment of the present disclosure.

FIG. 7 depicts an example 700 of an occupant activity that is relevant to indoor air quality.

### DETAILED DESCRIPTION

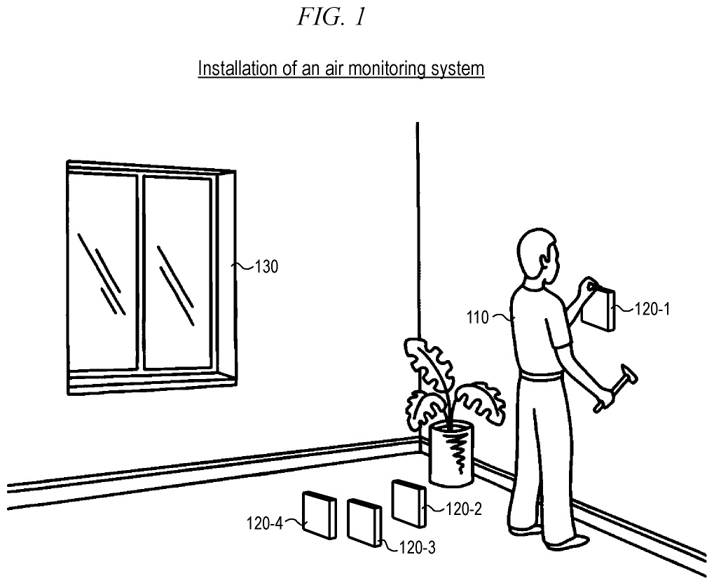

FIG. 1 depicts an aspect of an installation of an occupant-installed air monitoring system. In the figure, occupant 110 is depicted in the act of affixing an air quality sensor 120-1 on a wall in an indoor environment. In the depiction of FIG. 1, the air monitoring system that is being installed comprises multiple air quality sensors. The figure also depicts air quality sensors 120-2, 120-3, and 120-4, which have not yet been affixed to a wall.

In this disclosure, the words “air quality sensor” are used to refer to a unit that comprises one or more sensing devices capable of sensing one or more air quality parameters. Such a unit may also comprise additional capabilities. For example, air quality sensor 120-1 might comprise a communication capability for communicating sensed air quality parameters to a central unit or to other air quality sensors such as air quality sensors 120-2, 120-3, or 120-4.

A simple implementation of an air monitoring system might comprise a single unit wherein all necessary capabilities are implemented. For example, the single unit might comprise one or more sensing devices as well as a processing capability, a human interface capability, and any other necessary capabilities for implementing the full functionalities of an air monitoring system.

Alternatively, an air monitoring system might comprise a central unit that communicates with air quality sensors such as air quality sensors 120-1, 120-2, 120-3, and 120-4. Communication between the central unit and the sensors can be, in general, bi-directional, whereby the air quality sensors can communicate sensed air quality parameters (hereinafter, sensor readings) and other data to the central unit, and the central unit can communicate commands or requests to the air quality sensors. In the example of the previous paragraph, the single unit would perform these and other functionalities of the central unit. Even in an air monitoring system that comprises multiple sensors and a central unit, the central unit might combine central unit capabilities with sensor capabilities. In other words, the functionalities of the central unit might be performed by one of the sensor units, or even by a plurality of sensor units in a collaborative or distributed architecture.

Alternatively, an air monitoring system can comprise a remote implementation of one or more of its functionalities. For example, some or all of the functionalities of the central unit might reside in a remote server that is connected to the air quality sensors via one of many data communication technologies well known in the art. In such a remote implementation of part of the air monitoring system, the server might be implemented as a physical processor equipped with a physical memory, or it might be implemented as a distributed processor based on a plurality of physical processors with physical memories. Such a distributed processor might be referred to as a virtual processor or cloud processor whose functionalities are performed by one or more shared physical processors with physical memories and/or physical storage devices.

Alternatively, an air monitoring system that comprises a remote implementation of one or more of its functionalities can realize such implementation via one or more general-purpose processors, equipped with memories; wherein the processors execute software that implements such functionalities. For example, a general-purpose processor might be a personal computer owned or used by an occupant, wherein the personal computer executes a program that has been provided by the provider of the air monitoring system and is, therefore, part of the air monitoring system. Or a general-purpose processor might be a smartphone, or a tablet, or another personal device owned or used by an occupant, wherein the smartphone, tablet, or personal device executes an app that has been provided by the provider of the air monitoring system and, therefore, the app is part of the air monitoring system.

In the example of FIG. 1, occupant 110 is installing air quality sensor 120-1 in accordance with instructions that have been communicated to occupant 110 by the air monitoring system. Such instructions might be communicated by the air monitoring system to occupant 110, for example, via a human-interface device. For example, the air monitoring system might comprise an app that runs on a smartphone owned or used by the occupant. The app can display, on the smartphone's display, instructions generated by the air monitoring system. Alternatively, the app can speak the instructions via the speech-generating capabilities of the smartphone.

Furthermore, the air monitoring system might be communicating instructions to the occupant via other electronic means. For example, the app might run on a device other than a smartphone. For example, the app might run on a tablet, or on a computer, or on a watch, or on a television set, or on a smart mirror, or on smart glasses, or on a smart appliance, or on another electronic device. The app can communicate with other parts of the air monitoring system via a variety of communication technologies well known in the art; for example, one such possible technology might be a wireless Wi-Fi link, or a wireless Bluetooth link, or a connection via the Internet through a server or without a server, or any other connection technology. Also, the instructions might be communicated to the occupant via e-mail, text messaging, instant messaging, social media, a smart speaker, a telephone call, synthesized voice, or another method for communicating information.

Additionally, the air monitoring system might comprise other types of devices equipped with a human interface capability; for example, the air quality sensor 120-1 might comprise a human interface device capable of communicating instructions, or the air monitoring system might comprise a control unit (not depicted in FIG. 1) that comprises a human interface device capable of communicating instructions. Such human interface devices might be, for example, a display screen, and/or a touchscreen, and/or a keyboard, and/or a keypad, and/or an audio interface, and/or another human interface device that can be used for communicating installation instructions to the occupant.

In some embodiments of the present disclosure, the instructions provide guidance to the occupant for installing air quality sensors in an arrangement intended to achieve a desired characterization of the indoor environment. For example, the instructions might direct occupant 110 to install air quality sensor 120-1 within a range of acceptable distances from window 130, and/or within a range of heights above the floor that are acceptable for achieving the desired characterization of the indoor environment.

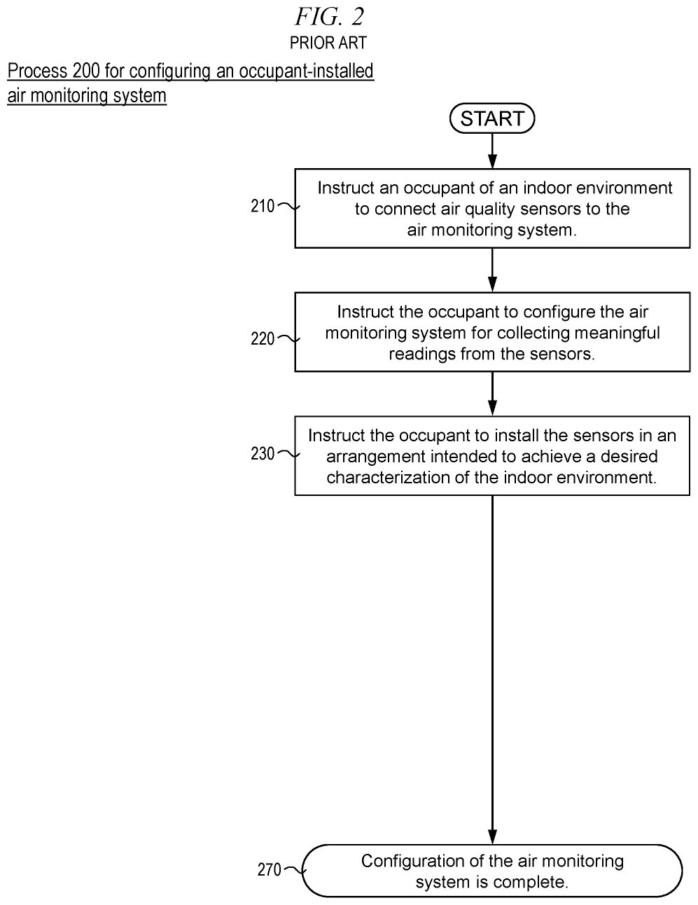

FIG. 2 is a flow diagram that illustrates a process 200, in the prior art, for configuring an occupant-installed air monitoring system. The process comprises tasks 210, 220, and 230. Block 270 represents the completion of the process.

Process 200 is for configuring an air monitoring system that comprises multiple air quality sensors. At task 210, an occupant of an indoor environment is instructed to connect the air quality sensors to the air monitoring system. Such connecting might be via a physical connection that might be implemented, for example, with one or more electrical wires, or with a local area network (LAN) that can be, for example, a wireless LAN.

After the air quality sensors are connected to the air monitoring system, task 220 can be executed. At task 220, the air monitoring system is configured; the objective of the configuration is to enable the air monitoring system to collect meaningful readings from the sensors. As part of task 220, the air monitoring system might, for example, receive information form the occupant about the location of some or all the sensors. For example, the air monitoring system might instruct the occupant to provide such information.

In FIG. 2, task 230 is depicted as following task 220; however, in some air monitoring systems in the prior art, some or all of task 230 might be performed in a different order relative to tasks 210 and 220. At task 230, the air monitoring system instructs the occupant to install the sensors in an arrangement intended to achieve a desired characterization of the indoor environment. If, at task 220, the air monitoring system receives information from an occupant about the location of some or all the sensors, the air monitoring system expects that the arrangement accurately reflects such information. Accordingly, after the completion of tasks 210, 220, and 230, the air monitoring system proceeds to block 270, where the configuration of the air monitoring system is regarded as being complete.

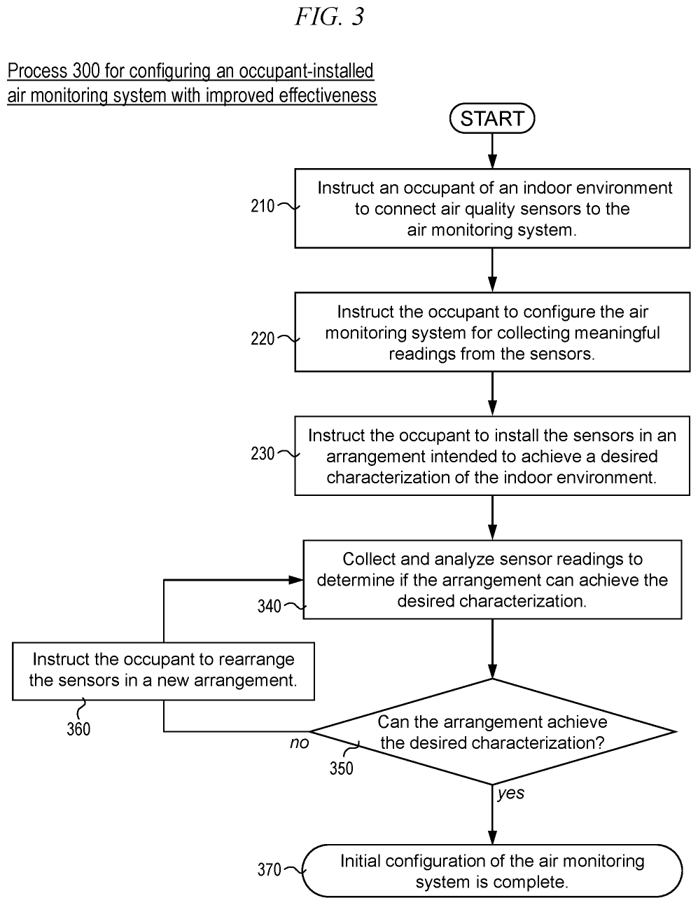

FIG. 3 is a flow diagram that illustrates a process 300 in accordance with a first illustrative embodiment of the present disclosure. The process is for configuring an occupant-installed air monitoring system. The process achieves a configuration of the air monitoring system whose effectiveness is improved, compared to prior-art occupant-installed air monitoring systems, and even compared to some prior-art expert-installed air monitoring systems.

Process 300 comprises tasks 210, 220, and 230, which are identical to tasks 210, 220, and 230 of FIG. 2. However, in contrast to the prior art, after completing these three tasks, the configuration of the air monitoring system is not regarded as being complete in process 300. Instead, process 300 continues with task 340. At task 340, the air monitoring system collects and analyzes sensor readings from one or more air quality sensors.

It will be clear to those skilled in the art, after reading this disclosure, how to make and use embodiments of the present disclosure wherein a variety of air quality sensors are used. An air quality sensor is a device that senses, detects, quantifies, and/or measures one or more parameters relevant to assessing air quality. There are many parameters that are relevant to assessing air quality, some of which may be directly related to air quality, while others may be related indirectly to air quality. For example, parameters directly related to air quality may include: air temperature, humidity, radiant temperature, operative temperature, pressure, ionizing radiation, air movement, suspended particulate matter, aerosols, volatile organic chemicals (VOC), reactive species, ozone, NOₓ, SOₓ, CO, CO₂, radicals, metals, and any other parameters that may be directly relevant to assessing air quality. For example, parameters indirectly related to air quality may include parameters such as room occupancy, occupant activity, lighting conditions, and sound conditions, which are known in the art to be relevant to assessing air quality. Furthermore, the status of doors and windows, including indoor doors, closet and furniture doors and, of course, doors to the outside, as well as the status of other openings to the outside, and other aspects of building status, are also known in the art to be relevant to assessing indoor air quality.

One might, for example, use a camera be coupled to an image processing system that detects and/or identifies occupant activity, or the status of doors, windows, furniture or other elements of an indoor environment. Such a camera and/or the image processing system can be regarded as an air quality sensor.

One might also, for example, use a temperature detector or an infrared camera to detect the status of an appliance, or a device, or an object whose status is characterized by a thermal signature. A system for detecting such type or similar status for an appliance, device, or object whose status affects air quality, directly or indirectly, can be regarded as an air quality sensor. For example, appliances, devices, or objects whose operation is characterized by a thermal signature comprise, of course, ovens, room heaters, hair dryers, hair curlers, fireplaces, candles, incandescent light bulbs, chafing canisters or dishes, room fragrance diffusers, or any other appliances, devices, or objects wherein temperature change is part of their desired functionality. However, there are many other appliances, devices, or objects where temperature change is incidental to their operation; the status of such appliances, devices, or objects can also affect air quality, for example, by affecting air motion. The status of such appliances, devices, or objects might also be detectable by detecting a thermal signature. For example, the status of vacuum cleaners, filtration devices, spray cans, computers, television sets, power tools, medical devices, and other appliances, devices, or objects can often be characterized by detecting a thermal signature. Indeed, the status of animals and people can often also be characterized by detecting a thermal signature. All of the abovementioned appliances, devices, or objects are known in the art to affect air quality, directly or indirectly. Many others are well known in the art to affect air quality. Any system that is capable of detecting aspects of the status of these and other appliances, devices or objects that affect air quality, by thermal or other means, can be regarded as an air quality sensor.

At task 340, the collected sensor readings are analyzed, by the air monitoring system, in order to determine if the arrangement of the sensors can achieve the desired characterization that was the objective of task 230. At task 350, a decision is made regarding whether or not the arrangement can achieve the desired characterization. If the air monitoring system decides that the arrangement cannot achieve the desired characterization, process 300 proceeds to task 360 next.

At task 360, the air monitoring system instructs one or more occupants to rearrange one or more of the sensors in a new arrangement. In particular, the air quality system communicates instructions, to the one or more occupants, for realizing the new arrangement. The instructions are based on the results of the analysis of the sensor readings performed as part of task 340. The objective of the new arrangement is to improve the effectiveness of the air monitoring system, so as to approach, as much as realistically possible, the desired characterization. After task 360, task 340 is executed again.

In this first illustrative embodiment of the present disclosure, tasks 340, 350, and 360, are repeated iteratively until the arrangement of sensors is deemed acceptable by the air monitoring system. It will be clear to those skilled in the art, after reading this disclosure, how to make and use alternative embodiments of the present disclosure wherein different types of criteria for acceptability are used. For example, alternative embodiments of the present disclosure can modify the desired characterization on the basis of the results of the analysis of sensor readings performed at task 340 so that the arrangement of sensors is deemed to have achieved the desired characterization. Alternative embodiments of the present disclosure can also decide to terminate the iterations of tasks 340, 350, and 360, based on one or more criteria other than achievement of a desired characterization.

In process 300, at task 350, if a decision is made that the arrangement can achieve the desired characterization, process 300 proceeds to block 370 which represents the completion of an initial configuration of the air monitoring system. Because of the multiple iterations of tasks 340, 350, and 360, the effectiveness of the air monitoring system is improved, compared to a prior-art air monitoring system that does not comprise such iterations. In some embodiments of the present disclosure, the effectiveness of the air monitoring system, as achieved via this initial configuration, might be regarded as sufficient without requiring additional improvements. In other embodiments of the present disclosure, the effectiveness of the air monitoring system can be further improved, as illustrated below in this disclosure.

In some small-size embodiments of the present disclosure, an entire air monitoring system might comprise only a single unit. That single unit might comprise a single sensor for just one air quality parameter, or it might comprise multiple sensors for one or more air quality parameters. Such embodiments might be especially advantageous, for example, in a small apartment or in a single-room type of indoor environment. In such single-unit embodiments, the single unit needs to be positioned in a suitable position and/or orientation within the indoor environment. Such a unit might be a stand-alone air monitoring system, or it might implement the full functionality of an air monitoring system in conjunction with a remote processing device such as a user device like a smartphone or tablet or other type of user device. Alternatively, the remote processing device might be implemented as a remote computer or cloud processor. In such embodiments, the remote processing device might be running an app that is part of the air monitoring system.

It is to be understood that, in embodiments of the present disclosure that comprise a single unit with one or more sensors, the expression “arrangement of sensors” or similar expressions should be understood to refer to the positioning of the single unit within the indoor environment, and to the orientation of the single unit relative to the indoor environment. The iterative sequence of tasks 340, 350, and 360 is still meaningful because the positioning and orientation of a single unit within an indoor environment has a significant influence on its effectiveness at characterizing indoor air quality. Indeed, even in embodiments that comprise multiple sensor units, the orientations of such units, as well as their positions relative to one another and to the indoor environment, are all parts of what is regarded as the arrangement of the sensors.

This first illustrative embodiment of the present disclosure is presented solely for illustration purposes. It will be clear to those skilled in the art, after reading this disclosure, how to make and use embodiments of the present disclosure that comprise some or all of the various illustrative alternatives presented in this disclosure, and/or other variations, while conforming to the definition of the present disclosure as set forth in the claims.

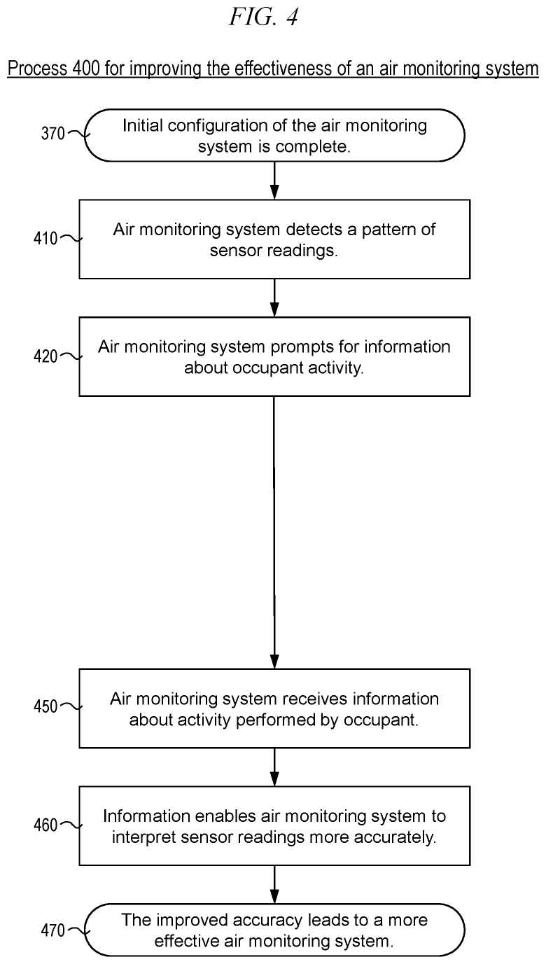

FIG. 4 is a flow diagram that illustrates a process 400 in accordance with a second illustrative embodiment of the present disclosure. The process comprises the same tasks and blocks as process 300, with the addition of tasks 410, 420, 450, and 460, and block 470.

After block 370, process 400 proceeds to task 410 wherein the air monitoring system detects a pattern of sensor readings. For example, the pattern might comprise one or more sensor readings that are different in character from sensor readings collected previously. Such difference might be associated with a particular activity that has been initiated by one or more occupants. For example, cooking a meal might cause a temperature sensor to yield a temperature reading that is higher than prior to the start of the cooking activity.

There is a body of knowledge in the art regarding correlations between air quality and cooking. There is also a body of knowledge in the art regarding correlations between air quality and many other activities. If the air monitoring system is informed about what activity is occurring in association with the detected pattern, the abovementioned body of knowledge can be exploited, by the air monitoring system, to further improve its effectiveness. Accordingly, process 400 proceeds to task 420 wherein the air monitoring system issues a prompt for information about occupant activity. For example, the air monitoring system might send a text message to an occupant with a request for information about any activities that might be going on.

Although, in the previous paragraph, the air monitoring system prompts for information about occupant activity via a text message, it will be clear to those skilled in the art, after reading this disclosure, how to make and use embodiments of the present disclosure wherein other methods are used for prompting. For example, prompting might be via an app that is installed in a smartphone, or in a tablet, or in a computer, or in a watch, or in a television set, or in a smart mirror, or in smart glasses, or in a smart appliance, or in another electronic device. Prompting might also be via e-mail, instant messaging, social media, a smart speaker, a telephone call, synthesized voice, an alert such as a buzzer or a blinking light, or another method for requesting information.

A prompt might be a simple open-ended prompt, or it might have the format of a multiple-choice prompt wherein a respondent can select one out of a plurality of multiple choices, as is well known in the art. Multiple hierarchical forms of such multiple-choice prompts are also well known in the art, as are many other formats for issuing a prompt that can be utilized in embodiments of the present disclosure.

One example of an occupant activity that might be detected by an air monitoring system is the case of an indoor environment where construction workers are engaging in construction or remodeling activities. In such a case, an air monitoring system might detect a pattern of sensor readings possibly associated with the use of certain building materials. The air monitoring system can prompt the occupants, which in this case include the construction workers, for information about the type of construction materials and techniques that have been used, the date of installation, the age and source of the materials, and/or any other relevant information that might be available. The air monitoring system can then exploit the available body of knowledge regarding correlations between air quality and these and other construction materials and techniques.

Although, in the preceding paragraphs, the second illustrative embodiment of the present disclosure has been described as prompting an occupant for information, it will be clear to those skilled in the art, after reading this disclosure, how to make and use embodiments of the present disclosure wherein the air monitoring system prompts others for information about occupant activity, instead of prompting an occupant. Indeed, in the example of the previous paragraph, it is the construction workers that are the occupants that are performing the construction activities; however, it might be better to prompt a manager of the construction workers for information about construction materials and techniques involved in the activity, rather than the construction workers themselves.

Although in this second illustrative embodiment of the present disclosure the air monitoring system prompts for information as a consequence of detecting a pattern of sensor readings, as set forth in task 410, it will be clear to those skilled in the art, after reading this disclosure, how to make and use embodiments of the present disclosure wherein the air monitoring system prompts for information for other reasons. For example, in an alternative embodiment of the present disclosure, an air monitoring system might prompt for information about occupant activities at a specific time of day known in advance to be often associated with certain activities. For example, an air monitoring system might prompt an occupant with a question about dinner at a typical dinner time. Alternatively, for example, an air monitoring system might have access to information from social media, such as a picture of food, that suggests the occurrence of an activity, such as cooking, that is relevant to air quality; in such a case, the air monitoring system might prompt an occupant for confirmation of the occurrence of the activity. In an alternative embodiment, an air monitoring system might, for example, have access to the sounds collected by a smart speaker and, based on an analysis of those sounds, it might be possible, for the air monitoring system, to conclude that a particular activity might be occurring; in such a case, the air monitoring system might prompt an occupant for confirmation of the occurrence of the activity.

In the second illustrative embodiment of the present disclosure, process 400, after prompting for information about an activity at task 420, proceeds to task 450 where the air monitoring system receives information about the activity. It then proceeds to task 460, where the availability of the information enables the air monitoring system to interpret sensor readings more accurately. Block 470 represents the completion of the process whereby the improved accuracy leads to a more effective air monitoring system.

This second illustrative embodiment of the present disclosure is presented solely for illustration purposes. It will be clear to those skilled in the art, after reading this disclosure, how to make and use embodiments of the present disclosure that comprise some or all of the various illustrative alternatives presented in these paragraphs, and/or other variations, while conforming to the definition of the present disclosure as set forth in the claims.

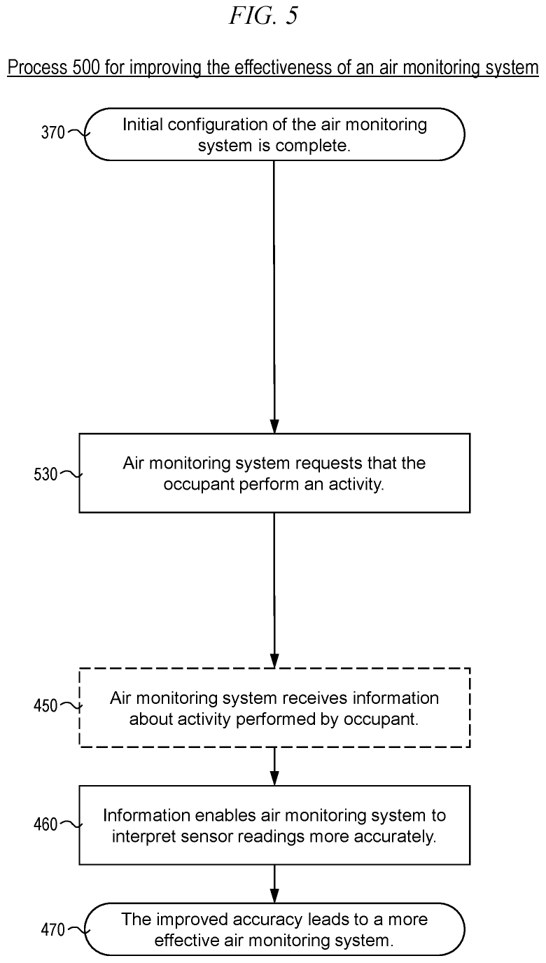

FIG. 5 is a flow diagram that illustrates a process 500 in accordance with a third illustrative embodiment of the present disclosure. The process comprises the same tasks and blocks as process 300, with the addition of tasks 530, 450, and 460, and block 470.

After block 370, process 500 proceeds to task 530 wherein the air monitoring system requests that the occupant perform an activity. This is in contrast to the second illustrative embodiment wherein the air monitoring system prompted an occupant for information about an activity that was occurring without any inducement by the air monitoring system. In this third illustrative embodiment, the air monitoring system explicitly submits a request that one or more occupants engage in a specific activity, or in a specific type of activity. For example, an air monitoring system might determine that its effectiveness could be improved by learning about what happens to sensor readings when occupants engage in vigorous physical activity. The air monitoring system can then submit a request to one or more occupants that they engage in such an activity.

Activities that might be requested also include, for example, opening or closing doors or windows, or changing the status of other structural elements of the environment such as vents, air exhaust openings, furniture, wall coverings, draperies, screens, shutters, blinds, tapestries, rugs, floor coverings, or other structural elements of the environment. Other activities that might be requested comprise activities that might also occur or have occurred in the past without inducement by the air monitoring system such as activities that have been mentioned in previous paragraphs. Indeed, the air monitoring system might simply request that one or more occupants continue performing one or more activities that are already ongoing. For example, the request might be that occupant or occupants keep windows open for a certain period of time, or until a later request that windows be closed.

Alternatively, a requested activity might actually be the cessation of another activity. Such a request might be submitted even if the nature of the other activity is not explicitly known to the air monitoring system. For example, an air monitoring system might be performing a collection and analysis of sensor readings for a purpose unrelated to occupant activities. For example, the purpose might be sensor calibration. It might be problematic if such a collection is corrupted by extraneous or unrelated data. If, at the same time, an occupant starts, for example, using cleaning products, the air monitoring system might detect the presence of extraneous sensor readings due to the use of the cleaning products. The air monitoring system might then submit a request that the occupant cease the activity, even without knowledge of the nature of the activity that is causing the extraneous sensor readings.

The air monitoring system can submit a request via any of the methods and channels that were mentioned in previous paragraphs for prompting for information. Furthermore, it will be clear to those skilled in the art, after reading this disclosure, how to make and use embodiments of the present disclosure wherein a request that an activity be performed is submitted without requiring that it be fulfilled by a specific responder.

After completing task 530, process 500 can proceed, optionally, to task 450. This task is identical to task 450 of process 400. At this task, the air monitoring system optionally receives information about the requested activity. For example, such information might be provided by the occupant that fulfils the activity request, or by any other that is capable of providing such information. In some embodiments of the present disclosure, task 450 is not present and, after task 530, the next task is task 460.

After optionally performing task 450, process 500 proceeds to task 460 and block 470, which are identical to task 460 and block 470 of process 400. At task 460, the availability of the information enables the air monitoring system to interpret sensor readings more accurately. Block 470 represents the completion of the process whereby the improved accuracy leads to a more effective air monitoring system.

This third illustrative embodiment of the present disclosure is presented solely for illustration purposes. It will be clear to those skilled in the art, after reading this disclosure, how to make and use embodiments of the present disclosure that comprise some or all of the various illustrative alternatives presented in these paragraphs, and/or other variations, while conforming to the definition of the present disclosure as set forth in the claims.

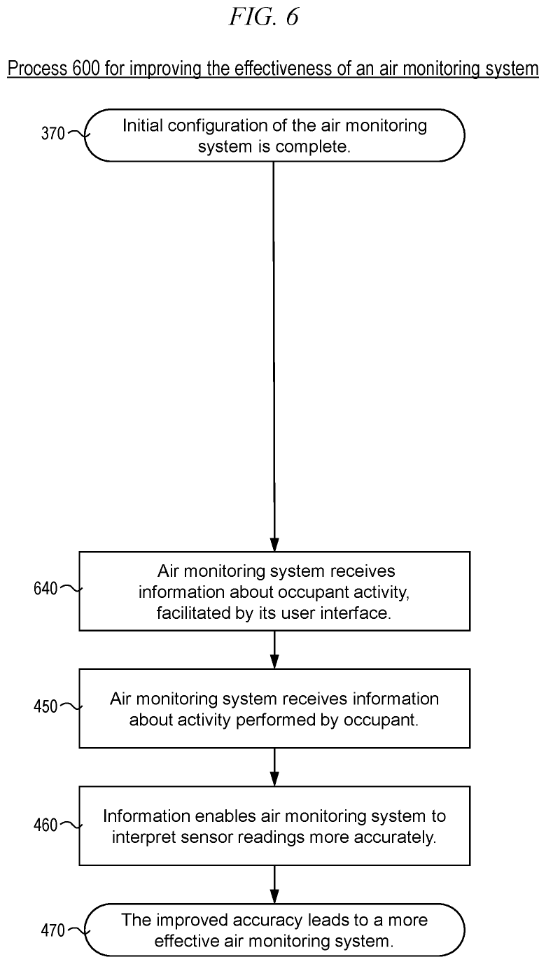

FIG. 6 is a flow diagram that illustrates a process 600 in accordance with a fourth illustrative embodiment of the present disclosure. The process comprises the same tasks and blocks as process 300, with the addition of tasks 640, 450, and 460, and block 470.

After block 370, process 600 proceeds to task 640 wherein the air monitoring system receives information about occupant activity. At task 640, the receiving of such information is facilitated by a user interface of the air monitoring system.

This is in contrast to the second illustrative embodiment wherein the air monitoring system prompted an occupant for information about an activity. In this fourth illustrative embodiment, the occupant is not prompted by the air monitoring system; instead, the occupant provides information without inducement from the air monitoring system. In this fourth illustrative embodiment, the providing of information by the occupant is facilitated by a user interface of the air monitoring system. For example, the user interface might be part of an app that is installed in a smartphone, or in a tablet, or in a computer, or in a smart watch, or in a television set, or in a smart mirror, or in smart glasses, or in a smart appliance, or in another electronic device. When the occupant wishes to communicate information about an activity, the occupant can activate the app and use it to communicate the information. In this illustrative embodiment, the app is the part of the air monitoring system that comprises a user interface that facilitates the receiving of the information by the air monitoring system.

It will be clear to those skilled in the art, after reading this disclosure, how to make and use embodiments of the present disclosure wherein the user interface is implemented by other means. For example, the receiving of information by the air monitoring system might be facilitated by a user interface that is based on one or more of e-mail, instant messaging, social media, a telephone call, voice recognition via a smart speaker or via another microphone-equipped device, a keyboard or keypad, knobs, pushbuttons, or another method for facilitating reception of information.

It will be clear to those skilled in the art, after reading this disclosure, that the activity about which the air monitoring system receives information in task 640 might have occurred prior to the receiving of the information; or it might be occurring simultaneously with the receiving of the information; or it might be an activity that is about to occur in the future or is planned for a future time, or is expected to occur at some point in the future; or the time of occurrence of the activity might be a combination of past, and/or present, and/or future.

Although, in this fourth illustrative embodiment of the present disclosure, the air monitoring system receives information from an occupant, it will be clear to those skilled in the art, after reading this disclosure, that the information can be received from individuals other than an occupant. Indeed, in the example of the construction workers presented in conjunction with the second illustrative embodiment of the present disclosure, a manager of the construction workers could be prompted for information about construction materials and techniques. In this fourth illustrative embodiment, the manager can voluntarily provide information via a user interface without being prompted.

This fourth illustrative embodiment of the present disclosure is presented solely for illustration purposes. It will be clear to those skilled in the art, after reading this disclosure, how to make and use embodiments of the present disclosure that comprise some or all of the various illustrative alternatives presented in these paragraphs, and/or other variations, while conforming to the definition of the present disclosure as set forth in the claims.

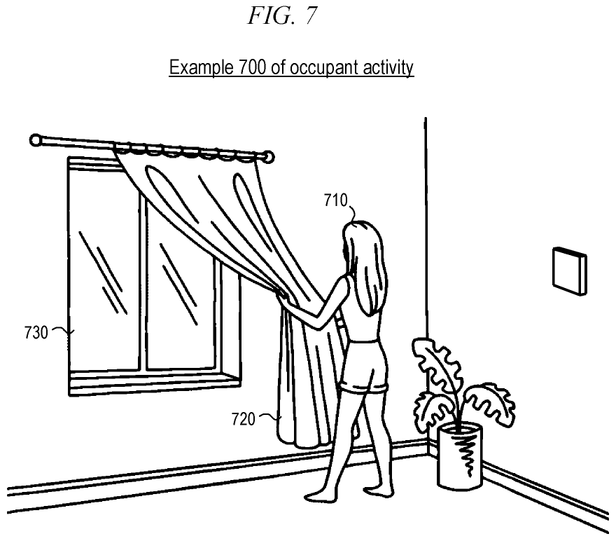

FIG. 7 depicts an example 700 of an occupant activity that is relevant to indoor air quality. In example 700, occupant 710 of an indoor environment is pushing aside draperies 720 away from window 730. This activity has the potential to affect indoor air quality in various ways. For example, the absence of draperies 720 in front of window 730 means that cold or heat from outside can more easily affect indoor temperature. Also, sunlight that might enter the indoor environment through a window is known to possibly affect indoor air quality; and the moving of the draperies might cause dust or allergens to be introduced into the indoor air.

Although the second, third, and fourth illustrative embodiments of the present disclosure have been presented as comprising the same tasks and blocks as process 300, it will be clear to those skilled in the art, after reading this disclosure, how to make and use alternative embodiments of the present disclosure that may not comprise tasks 340, and/or 350, and/or 360, but, otherwise, comprise the other tasks and blocks that are part of the second, or third, or fourth illustrative embodiment. The illustrative embodiments of the present disclosure presented in this disclosure are presented solely for illustration purposes. It will be clear to those skilled in the art, after reading this disclosure, how to make and use embodiments of the present disclosure that comprise some or all of the various illustrative alternatives presented in this disclosure, or other alternatives, while conforming to the definition of the present disclosure as set forth in the claims.

In this disclosure, illustrative embodiments of the present disclosure comprise various tasks that occur in various sequences. It will be clear to those skilled in the art, after reading this disclosure, how to make and use embodiments of the present disclosure that comprise some or all of those tasks, or other tasks, in similar or different sequences. In many cases, tasks can occur in different temporal sequences in different embodiments of the present disclosure that conform to the definition of the present disclosure as set forth in the claims.

Some air monitoring systems in the prior art comprise a capability for coaching occupants about how to improve air quality in an indoor environment. Those skilled in the art will understand that improving air quality is not the same as improving the effectiveness of an air monitoring system in the performance of its air monitoring functionality. Embodiments of the present disclosure can improve the effectiveness of an indoor air monitoring system through various types of interactions with occupants. Compared to prior-art air monitoring systems, embodiments of the present disclosure can achieve levels of effectiveness at characterizing indoor air quality that can surpass the effectiveness of prior-art air monitoring systems at such characterizing regardless of the actual air quality.

It will be understood by those skilled in the art that activities that are relevant to indoor air quality do not necessarily occur indoors. For example, changing a filter in an air conditioning unit that supplies air to an indoor environment is very relevant to indoor air quality even if the unit is located outdoors. Also, for example, starting or operating an automobile or a lawn mower or a grass trimmer or any device equipped with an internal combustion engine in the vicinity of an indoor environment such as a house or other type of building will, unavoidable, have an effect on indoor air quality, especially if the activity occurs near an air intake for the indoor environment. The same is true of using paint or other chemicals outdoors, if the activity occurs near the indoor environment.

It will be understood by those skilled in the art that activities that are relevant to indoor air quality do not necessarily always involve the release of a chemical or a substance that affects air quality. In many cases, an activity that results in the removal of a substance from the air can have an important effect on air quality and should be regarded as relevant to air quality. For example, replacing a filter in an air conditioner unit is an activity that is relevant to indoor air quality. Other examples of such activities include, for example, removal of building materials that contribute contaminants such as, for example, asbestos, formaldehyde, VOCs or other types of contaminants, or covering open containers, or turning off appliances, or other activities that result in the removal of a substance, or the removal of an object that emits a substance.

Some of the embodiments of the present disclosure benefit from the cooperation of human individuals in order to achieve one or more desired results. Gamification techniques are known in the art as being useful for increasing the probability of effective cooperation by human individuals. Gamification is the application of game-design elements and game principles in non-game contexts. Gamification commonly employs game design elements to improve one or more of: human engagement, cooperation, flow, learning, crowdsourcing, ease of use, usefulness of systems, and/or, more generally, to enhance the effectiveness of interactions of humans with systems. Some research studies on gamification show that it has positive effects on individuals.

Gamification techniques can leverage people's natural desires for socializing, learning, mastery, competition, achievement, status, self-expression, altruism, or closure, or simply leverage their response to the framing of a situation as game or play. Some gamification techniques provide rewards for individuals that accomplish desired task, or utilize competition to engage individuals. Types of rewards include, among others, points, achievement badges or levels, the filling of a progress bar, or providing the user with virtual or real currency. Making the rewards for accomplishing tasks visible to other players or individuals, or providing leader boards, are among many ways of encouraging players to compete through public disclosure of their accomplishments.

An important aspect of gamification is the social interaction with peers and/or other members of a community. Through the use social media, individual accomplishments yield recognition and approval, while failures yield strong pressure for change. In fact, even just the knowledge that others are participants is often sufficient to persuade an individual to get involved. This phenomenon is known in the art as FOMO (i.e., fear of missing out). Aspects of gamification such as these, and others, have been shown to be a powerful inducement toward greater and more effective involvement in a wide variety of situations.

An embodiment of the present disclosure can, for example, use gamification techniques as part of performing task 360. The air monitoring system can reward the occupant for his/her participation with badges, or points, or other types of recognition that can be publicized in social media and compared to similar rewards obtained by other occupants of other environments that are equipped with other air monitoring systems. Rewards can also be more tangible, such as, for example, discount coupons or monetary credit toward products or services related (or not related) to air quality, or related to air quality monitoring, or to something else.

Embodiments of the present disclosure can also, for example, use gamification techniques as part of performing task 420. For example, when prompting for information about occupant activity, the air monitoring system can display, along with a prompt, an indication of a score associated with responding to the prompt. The air monitoring system can, for example, award the score based on aspects of the response to the prompt. For example, a more timely response could yield a higher score than a less timely response. Also, the type of information received by the air monitoring system in response to the prompt can be evaluated by the air monitoring system to assess its usefulness. More useful information might yield a higher score than less useful information. Additionally, thoroughness of responding to prompts might be rewarded with higher scores. For example, extra bonus scores might be awarded when the number of responses to prompts reaches a certain target. Other gamification techniques, such as those mentioned elsewhere in this disclosure, and others, can also be used in performing task 420, as well as in performing other tasks.

Embodiments of the present disclosure can also, for example, use gamification techniques as part of performing task 530. For example, when requesting that an occupant perform an activity, the air monitoring system can provide information about the requested activity with the aim of increasing the odds that the activity, when performed, will provide useful data for the air monitoring system. Gamification techniques can greatly increase the odds of compliance by the occupant. For example, if the requested activity is a type of physical exercise, a report of calories that can be burned, along with comparisons with calories burned by peers doing similar exercises, as reported on social media, is a gamification technique that can be a powerful inducement to complying with the request. Furthermore, additional rewards can be provided if the occupant is successful in fulfilling the request in accordance with specific instructions provided by the air monitoring system, so that the effectiveness of the activity, at providing useful information to the air monitoring system, is enhanced. Real-time reports to the occupant about how the air monitoring system is affected by the occupant's activity can also be enhanced with gamification techniques to increase occupant interest, participation and compliance with requests.

Embodiments of the present disclosure can also, for example, use gamification techniques as part of performing task 640. For example, when information about occupant activity is received by the air monitoring system, as facilitated by the user interface of the air monitoring system, the air interface can provide rewards based on the quality and usefulness of the information provided. Such rewards might be as simple as visually pleasing and emotionally gratifying feedback that acknowledges the value of the information received. Also, rewards of various types, and reports on social media, and other types of encouragement based on gamification techniques can be incorporated in the design of the user interface. Furthermore, even before the air monitoring system receives information, the design of the user interface can be adapted to encourage the providing of information. For example, the air monitoring system can schedule events and/or competitions via social media, or it can provide access to events and/or competitions that are likely to enhance the odds of useful information being received via the user interface.

Gamification techniques are also useful for encouraging occupants to be more proactive and consistent. For example, a well-structured reminder based on gamification techniques can be used to remind an occupant to access the user interface whenever the occupant performs a particular activity. Also, gamification techniques can enhance instructions or directives that the air monitoring system conveys to an occupant about how a particular activity or type of activity ought to be performed.

It is to be understood that this disclosure teaches just one or more examples of one or more illustrative embodiments, and that many variations of the disclosure can easily be devised by those skilled in the art after reading this disclosure, and that the scope of the present disclosure is defined by the claims accompanying this disclosure.

1. A method of configuring an occupant-installed air monitoring system, the method comprising:
(i) communicating, by the air monitoring system, to a first occupant of an indoor environment, one or more arrangement instructions for arranging one or more air quality sensors in a first arrangement of the air quality sensors in the indoor environment;
(ii) collecting, by the air monitoring system, one or more readings from the one or more air quality sensors;
(iii) communicating, by the air monitoring system, to a second occupant of the indoor environment, one or more rearrangement instructions for rearranging the one or more air quality sensors in a second arrangement of the air quality sensors in the indoor environment;
wherein (ii) collecting occurs after (i) communicating and (iii) communicating occurs after (ii) collecting;
wherein the rearrangement instructions are generated by the air monitoring system based on the one or more readings from the one or more air quality sensors; and
wherein the first occupant and the second occupant are the same occupant or are not the same occupant.

2. The method of claim 1 further comprising:
(iv) receiving, by the air monitoring system, information about an activity performed by a third occupant;
wherein the third occupant is the same occupant as the first occupant, or is the same occupant as the second occupant, or both, or the third occupant is not the same occupant as either the first occupant or the second occupant.

3. The method of claim 2 further comprising:
(v) prompting, by the air monitoring system, for the information about the activity performed by the third occupant;
wherein (v) prompting occurs before (iv) receiving; and
wherein (v) prompting facilitates (iv) receiving.

4. The method of claim 3 wherein one or more of (i) communicating, or (iii) communicating, or (iv) receiving, or (v) prompting are enhanced via one or more gamification techniques.

5. The method of claim 2 further comprising:
(v) requesting, by the air monitoring system, that the third occupant perform the activity;
wherein (v) requesting occurs before (iv) receiving.

6. The method of claim 5 wherein one or more of (i) communicating, or (iii) communicating, or (iv) receiving, or (v) requesting are enhanced via one or more gamification techniques.

7. The method of claim 5 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

8. The method of claim 2 wherein (iv) receiving is facilitated by a user interface of the air monitoring system.

9. The method of claim 8 wherein one or more of (i) communicating, or (iii) communicating, or (iv) receiving, or the user interface are enhanced via one or more gamification techniques.

10. The method of claim 1 wherein the arrangement instructions, or the rearrangement instructions, or both are enhanced via one or more gamification techniques.

11. An apparatus for monitoring indoor air quality, the apparatus comprising:
(a) a processor,
(b) a memory,
(c) one or more air quality sensors,
(d) one or more user-interface devices;
wherein the memory stores executable processor instructions that, when executed by the processor, facilitate performance of operations, the operations comprising:
(i) communicating, to a first occupant of an indoor environment, one or more positioning instructions for positioning the apparatus in a first position in the indoor environment;
(ii) collecting one or more readings from the one or more air quality sensors;
(iii) communicating, to a second occupant of the indoor environment, one or more repositioning instructions for repositioning the apparatus in a second position in the indoor environment;
wherein (ii) collecting occurs after (i) communicating and (iii) communicating occurs after (ii) collecting;
wherein the repositioning instructions are generated by the apparatus based on the one or more readings from the one or more air quality sensors; and
wherein the first occupant and the second occupant are the same occupant or are not the same occupant.

12. The apparatus of claim 11 wherein the operations further comprise:
(iv) receiving, by the apparatus, information about an activity performed by a third occupant;
wherein the third occupant is the same occupant as the first occupant, or is the same occupant as the second occupant, or both, or the third occupant is not the same occupant as either the first occupant or the second occupant.

13. The apparatus of claim 12 wherein one or more of (i) communicating, or (iii) communicating, or (iv) receiving, or the positioning instructions, or the repositioning instructions are enhanced via one or more gamification techniques.

14. A method of configuring an occupant-installed air monitoring system, the method comprising:
(i) communicating, by the air monitoring system, to a first occupant of an indoor environment, one or more arrangement instructions for arranging one or more air quality sensors in a first arrangement of the air quality sensors in the indoor environment;
(ii) receiving, by the air monitoring system, information about an activity performed by a second occupant;
wherein the first occupant and the second occupant are the same occupant or are not the same occupant.

15. The method of claim 14 wherein one or more of (i) communicating, or (ii) receiving are enhanced via one or more gamification techniques.

16. The method of claim 14 further comprising:
(iii) communicating, by the air monitoring system, to a third occupant of the indoor environment, one or more rearrangement instructions for rearranging the one or more air quality sensors in a second arrangement of the air quality sensors in the indoor environment;
wherein the rearrangement instructions are generated by the air monitoring system based on the information about the activity performed by the second occupant; and
wherein the third occupant is the same occupant as the first occupant, or is the same occupant as the second occupant, or both, or the third occupant is not the same occupant as either the first occupant or the second occupant.

17. The method of claim 16 wherein one or more of (i) communicating, or (ii) receiving, or (iii) communicating are enhanced via one or more gamification techniques.

18. The method of claim 14 further comprising:
(iii) prompting, by the air monitoring system, for the information about the activity performed by the second occupant;
wherein (iii) prompting occurs before (ii) receiving; and
wherein (iii) prompting facilitates (ii) receiving.

19. The method of claim 14 further comprising:
(iii) requesting, by the air monitoring system, that the second occupant perform the activity;
wherein (iii) requesting occurs before (ii) receiving.

20. The method of claim 18 wherein (ii) receiving is facilitated by a user interface of the air monitoring system.

21. An occupant-installed air monitoring system for monitoring indoor air quality, the system comprising:
a processor, and a memory that stores executable processor instructions that, when executed, facilitate performance of operations, the operations comprising:
(i) communicating, by the air monitoring system, to a first occupant of an indoor environment, one or more arrangement instructions for arranging one or more air quality sensors in a first arrangement of the air quality sensors in the indoor environment;
(ii) collecting, by the air monitoring system, one or more readings from the one or more air quality sensors;
(iii) communicating, by the air monitoring system, to a second occupant of the indoor environment, one or more rearrangement instructions for rearranging the one or more air quality sensors in a second arrangement of the air quality sensors in the indoor environment;
wherein (ii) collecting occurs after (i) communicating and (iii) communicating occurs after (ii) collecting;
wherein the rearrangement instructions are generated by the air monitoring system based on the one or more readings from the one or more air quality sensors; and
wherein the first occupant and the second occupant are the same occupant or are not the same occupant.

22. The air monitoring system of claim 21 wherein the operations further comprise:
(iv) receiving, by the air monitoring system, information about an activity performed by a third occupant;
wherein the third occupant is the same occupant as the first occupant, or is the same occupant as the second occupant, or both, or the third occupant is not the same occupant as either the first occupant or the second occupant.

23. The air monitoring system of claim 22 wherein the operations further comprise:
(v) prompting, by the air monitoring system, for the information about the activity performed by the third occupant;
wherein (v) prompting occurs before (iv) receiving; and
wherein (v) prompting facilitates (iv) receiving.

24. The air monitoring system of claim 23 wherein one or more of (i) communicating, or (iii) communicating, or (iv) receiving, or (v) prompting are enhanced via one or more gamification techniques.

25. The air monitoring system of claim 22 wherein the operations further comprise:
(v) requesting, by the air monitoring system, that the third occupant perform the activity;
wherein (v) requesting occurs before (iv) receiving.

26. The air monitoring system of claim 25 wherein one or more of (i) communicating, or (iii) communicating, or (iv) receiving, or (v) requesting are enhanced via one or more gamification techniques.

27. The air monitoring system of claim 25 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

28. The air monitoring system of claim 22 further comprising a user interface;
wherein the user interface facilitates (iv) receiving.

29. The air monitoring system of claim 28 wherein one or more of (i) communicating, or (iii) communicating, or (iv) receiving, or the user interface are enhanced via one or more gamification techniques.

30. The air monitoring system of claim 21 wherein the arrangement instructions, or the rearrangement instructions, or both are enhanced via one or more gamification techniques.

31. An occupant-installed air monitoring system for monitoring indoor air quality, the system comprising:
a processor, and a memory that stores executable processor instructions that, when executed, facilitate performance of operations, the operations comprising:
(i) communicating, by the air monitoring system, to a first occupant of an indoor environment, one or more arrangement instructions for arranging one or more air quality sensors in an arrangement of the air quality sensors in the indoor environment;
(ii) receiving, by the air monitoring system, information about an activity performed by a second occupant;
wherein the first occupant and the second occupant are the same occupant or are not the same occupant.

32. The air monitoring system of claim 31 wherein one or more of (i) communicating, or (ii) receiving are enhanced via one or more gamification techniques.

33. The air monitoring system of claim 31 wherein the operations further comprise:
(iii) communicating, by the air monitoring system, to a third occupant of the indoor environment, one or more rearrangement instructions for rearranging the one or more air quality sensors in a second arrangement of the air quality sensors in the indoor environment;
wherein the rearrangement instructions are generated by the air monitoring system based on the information about the activity performed by the second occupant; and
wherein the third occupant is the same occupant as the first occupant, or is the same occupant as the second occupant, or both, or the third occupant is not the same occupant as either the first occupant or the second occupant.

34. The air monitoring system of claim 33 wherein one or more of (i) communicating, or (ii) receiving, or (iii) communicating comprise one or more gamification techniques.

35. The air monitoring system of claim 31 wherein the operations further comprise:
(iii) prompting, by the air monitoring system, for the information about the activity performed by the second occupant;
wherein (iii) prompting occurs before (ii) receiving; and
wherein (iii) prompting facilitates (ii) receiving.

36. The air monitoring system of claim 31 wherein the operations further comprise:
(iii) requesting, by the air monitoring system, that the second occupant perform the activity;
wherein (iii) requesting occurs before (ii) receiving.

37. The air monitoring system of claim 35 wherein (ii) receiving is facilitated by a user interface of the air monitoring system.

38. The method of claim 1 wherein one or more of (i) communicating, or (iii) communicating are enhanced via one or more gamification techniques.

39. The method of claim 6 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

40. The method of claim 1 wherein the arrangement instructions, or the rearrangement instructions, or both are enhanced via one or more gamification techniques.

41. The air monitoring system of claim 21 wherein one or more of (i) communicating, or (iii) communicating are enhanced via one or more gamification techniques.

42. The air monitoring system of claim 26 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

43. The air monitoring system of claim 21 wherein the arrangement instructions, or the rearrangement instructions, or both are enhanced via one or more gamification techniques.

44. The apparatus of claim 12 wherein the operations further comprise:
(v) prompting, by the air monitoring system, for the information about the activity performed by the third occupant;
wherein (v) prompting occurs before (iv) receiving; and
wherein (v) prompting facilitates (iv) receiving.

45. The apparatus of claim 44 wherein one or more of (i) communicating, or (iii) communicating, or (iv) receiving, or (v) prompting are enhanced via one or more gamification techniques.

46. The apparatus of claim 12 wherein the operations further comprise:
(v) requesting, by the air monitoring system, that the third occupant perform the activity;
wherein (v) requesting occurs before (iv) receiving.

47. The apparatus of claim 46 wherein one or more of (i) communicating, or (iii) communicating, or (iv) receiving, or (v) requesting are enhanced via one or more gamification techniques.

48. The apparatus of claim 12 further comprising a user interface;
wherein the user interface facilitates (iv) receiving.

49. The apparatus of claim 48 wherein one or more of (i) communicating, or (iii) communicating, or (iv) receiving, or the user interface are enhanced via one or more gamification techniques.

50. The apparatus of claim 11 wherein one or more of (i) communicating, or (iii) communicating are enhanced via one or more gamification techniques.

51. The apparatus of claim 46 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

52. The apparatus of claim 11 wherein the arrangement instructions, or the rearrangement instructions, or both are enhanced via one or more gamification techniques.

53. The method of claim 19 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

54. The air monitoring system of claim 36 wherein the activity is an activity that modifies the indoor environment, and wherein the modifying comprises one or more of: changes to the structure of the environment, or application or removal of a substance, or a biological activity in the environment, or use of an appliance or furniture, or combustion of a combustible material, or an activity that results in a change in air quality.

55. The method of claim 14 wherein one or more of (i) communicating, or (iii) communicating are enhanced via one or more gamification techniques.

56. The air monitoring system of claim 31 wherein one or more of (i) communicating, or (iii) communicating are enhanced via one or more gamification techniques.
